(19) United States
(12) Patent Application Publication
(10) Pub. No.: US 2020/0316542 A1
(43) Pub. Date: Oct. 8, 2020

(54) TATTOO INK CUSTOM MIXING STATION

(71) Applicant: Ink Projects LLC, Fort Mill, SC (US)

(72) Inventor: Louis Rubino, JR., York, SC (US)

(73) Assignee: Ink Projects LLC, Fort Mill, SC (US)

(21) Appl. No.: 16/841,356

(22) Filed: Apr. 6, 2020

Related U.S. Application Data
(60) Provisional application No. 62/830,103, filed on Apr. 5, 2019.

(51) Int. Cl. B01F 13/10; B01F 15/02; B01F 3/08

(52) U.S. Cl. CPC B01F 13/1066; B01F 15/0243; B01F 3/088; A61M 37/0084; B01F 13/1063; B01F 2215/0059; B01F 3/0861

(57) ABSTRACT

Systems, devices and techniques are disclosed herein which includes a plurality of ink containers, each including a user-activated dispenser, each user-activated dispenser configured to dispense ink in a preselected amount related to a single activation of the dispenser. An input device is configured to receive a color input from a user, a first ink container from the plurality of ink containers comprising first colored ink and a first mixing mechanism configured to mitigate settlement of the first colored ink or otherwise agitate the ink. A second ink container from the plurality of ink containers includes second colored ink, and a second mixing mechanism is configured to mitigate settlement of the second colored ink or otherwise agitate the ink. A mixing container is configured to receive at least the first colored ink from the first container and the second colored ink from the second container in an amount as and to store custom ink.

### BACKGROUND

The tattoo industry has become highly creative, with tattoo artists constantly creating new and unique designs. The present application is directed to a system for allowing tattoo and/or permanent makeup artists to create unique and customized tattoo inks or permanent makeup inks to expand their scope of expression.

### SUMMARY

A system and devices are disclosed herein which includes a mixing station for customizable tattoo inks. As used herein, “tattoo ink” or “ink” refers to inks or dyes for coloring the skin, and includes, but is not limited to, tattoo inks and inks or dyes for permanent makeup. A mixing station generally comprises a workbench such as a table area, and a plurality of ink containers. The ink containers comprise user-activated dispensers, each user-activated dispenser configured to dispense ink in a preselected amount related to a single activation of the dispenser. The ink containers preferably contain tattoo inks of different colors. An input device is configured to receive a color input from a user, a first ink container from the plurality of ink containers comprising a first colored ink, and a first mixing mechanism configured to mitigate settlement of the first colored ink or otherwise agitate the ink. A second ink container from the plurality of ink containers includes a second colored ink, and a second mixing mechanism is configured to mitigate settlement of the second colored ink or otherwise agitate the ink.

Also disclosed is a mixing container configured to receive at least the first colored ink from the first container in an amount as selected by a user activating the user-activated dispenser a selected number of times, and the second colored ink from the second container in an amount as selected by a user activating the user-activated dispenser a selected number of times, and to store a custom ink. The mixing container may also comprise a mixing mechanism which may be the same as or different than the first mixing mechanism and the second mixing mechanism.

### BRIEF DESCRIPTION OF THE DRAWING(S)

The drawings described below are for illustration purposes only. The drawings are not intended to limit the scope of the present disclosure. Like reference characters shown in the figures designate the same parts in the various embodiments.

FIG. 1 is a diagram of a mixing station with a plurality of ink containers and a mixing container; and

FIG. 2 is a diagram of another mixing station with a plurality of ink containers, which provide ink into a mixing container.

FIG. 3 is a diagram of a mixing station with a plurality of alternate ink containers and a mixing container;

FIG. 4 is a diagram of another mixing station with a plurality of alternate ink containers, which provide ink into a mixing container;

FIG. 5 is a block diagram of an implementation of the disclosed embodiments; and

FIG. 6 is a diagram of an input selection mechanism.

### DETAILED DESCRIPTION OF THE PREFERRED EMBODIMENT(S)

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present invention. As used herein, the term “and/or” includes any and all combinations of one or more of the associated listed items.

The system and devices disclosed herein may enable the creation of a customized ink color while using a mixing station. The mixing station may include multiple ink containers, which contain different ink colors. The ink containers may be configured such that they include a user-activated dispenser that dispenses ink into a mixing container. The user-activated dispenser may dispense a preselected amount of ink per single activation of the dispenser. The user-activated dispenser may be any applicable dispenser that dispenses ink from an ink container, based on user provided input. A user may provide color input using an input device such as a button, knob, touch screen input, dial, lever, mouse, keyboard, switch or the like. Ink containers may include a mixing mechanism that mitigates settlement of the ink within the ink containers. The mixing mechanism may be, for example, a mixer (either mechanical or electric), a blender, a rod, a plate, a ball bearing, or any other component that mitigates settlement and/or agitates the ink. The mixing mechanism may move constantly or at intervals, to prevent settlement of the ink and/or to provide even mixing.

A mixing container may receive at least two colored inks from at least two ink containers such that the two colored inks mix to produce a custom ink color. As an example, a user may select 3 pump at a first ink container to dispense 3 ml of a first color and may also select 1 pump at a second ink container to dispense 1 ml of a second color. A mixing container may receive the 3 pumps of the first color and 1 pump of the second container to produce a custom ink with a 3:1 ratio of the first color in to the second color ink. The mixing container may also include a mixing mechanism configured to prevent settlement of the ink within the mixing container.

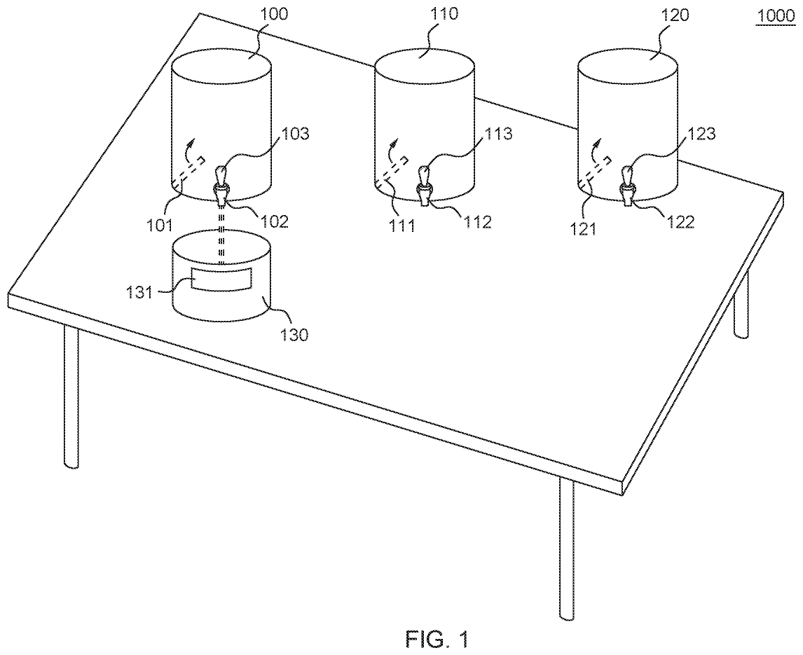

FIG. 1 shows an example mixing station 1000 comprising three ink containers 100, 110, and 120. The ink containers 100, 110, and 120 may be mounted or placed on a work surface 1100. The ink containers 100, 110, and 120 may each contain a different ink color and/or pigment. For example, ink container 100 may include red ink, ink container 110 may include green ink, and ink container 120 may include blue ink. Each of the ink containers 100, 110 and 120 may include a mixing mechanism 101, 111, and 121 respectively. The mixing mechanisms 101, 111, 121 may generally comprise blenders, agitators, such as moveable agitating arms or paddles, or the like. As shown, the mixing mechanisms 101, 111, and 121 may be located inside the ink containers 100, 110, and 120 and may rotate in the direction shown in FIG. 1. The agitation, rotation or other movement of the mixing mechanism s 101, 111, and 121 may mitigate the settlement of the ink contained in each respective ink container, and may be used to mix the ink to maintain the proper colors or consistency of custom ink such as by blending the ink. The mixing mechanism s 101, 111, and 121 may be mechanically or electrically operated blenders, having motors and blades for stirring the ink. A user may interact with input device buttons 103, 113, and/or 123 in order to dispense respective inks from the ink containers 100, 110, and 120. For example, a user may press button 103 to dispense 1 ml of red ink from the ink container 100 into the mixing container 130. The user may subsequently press button 123 three times to displease 3 ml of blue ink from the ink container 120 into the mixing container 130. The 1 ml of red ink may mix with the 3 ml of blue ink to produce a custom ink color within the mixing station. The ink may be dispensed from the ink containers 100, 110, and 120 via user-activated dispensers 102, 112, and 122, respectively. As shown in the example of FIG. 1, the user-activated dispenser may be configured to dispense ink into a container 130. It is appreciated that the user-activated dispensers 102, 112, and 122 can be buttons, valves, levers, or any other actuation devices allowing for the selective dispensing of the inks. The resultant tattoo ink may be referred to as a custom ink.

As further disclosed herein, a label 131 may be generated and may include a custom color and other indicia for the custom ink and/or a custom formula for the custom ink within the mixing container 130. The label (e.g., printout) may be printed at the mixing station 1000 or may be provided to the user via an alternative mode such as via email or via transmission to a printer separate from mixing station 1000. According to an implementation, the custom color, other indicia, and/or custom formula may be transmitted via an electronic transmission and may be stored and/or associated with a user profile.

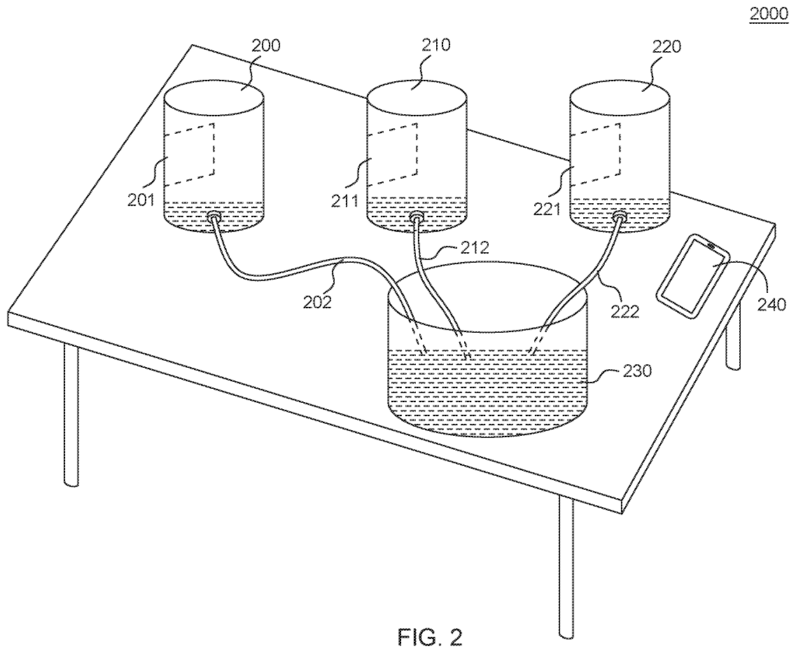

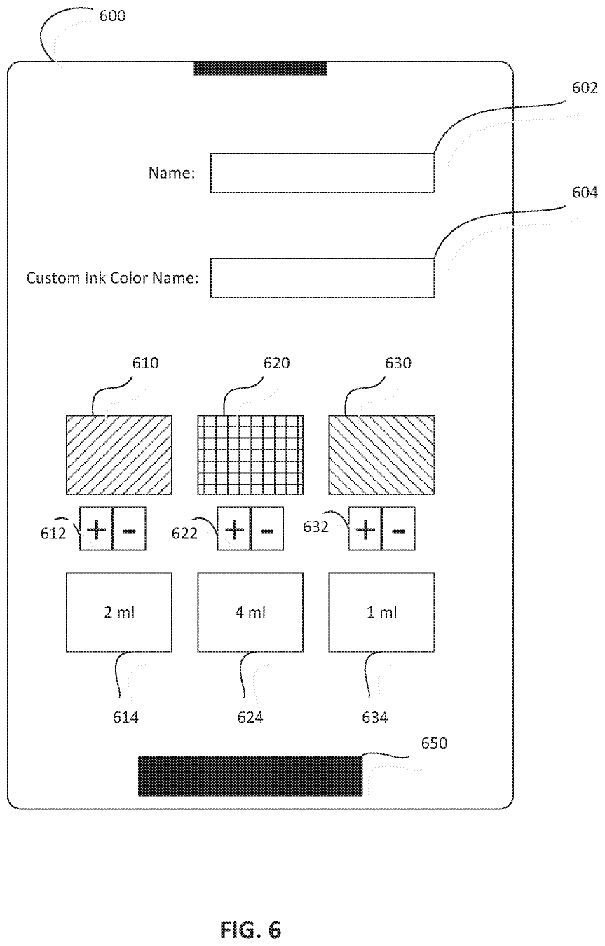

FIG. 2 shows another example mixing station 2000 comprising three ink containers 200, 210, and 220. The ink containers 200, 210, and 220 may be on a station 2100. The ink containers 200, 210, and 220 may each contain a different ink color. For example, ink container 200 may include red ink, ink container 210 may include green ink, and ink container 220 may include blue ink. Each of the ink containers 200, 210, and 220 may include a mixing mechanism 201, 211, and 221 respectively. As shown, the mixing mechanisms 201, 211, and 221 may be located inside the ink containers 200, 210, and 220 and may be attached to the walls of the ink containers. In this example, the walls of the ink containers 200, 210, and 220 may rotate such that the mixing mechanisms 201, 211, and 221 rotate along with the rotation of the walls. The movement such as rotation of the mixing mechanism 201, 211, and 221 may mitigate the settlement of the ink contained in each respective ink container. A user may interact with input device 240, which may be a mobile device, a tablet, a notebook, a laptop, or the like and may enable interaction via a touch screen, keyboard, mouse or other device. The input device 240 may be a mobile device, wireless device, computer, tablet, notebook, or similar computerized device, and may comprise a software application configured to interact with a user and the mixing stations to allow for the selection, adjustment, and customization of the various inks to arrive at a custom ink. FIG. 6, as further disclosed herein, is an example of an input device. Interaction with the input device 240 may cause ink to dispense from one or more of the ink containers 200, 210, and 220, as input by a user into the input device 240. For example, a user may input a ratio of 3:2:4 corresponding to 3 parts red, 2 parts green, and 4 parts blue via the input device 240. Based on the user input, a signal or other indication may be provided to ink container 200 to dispense 3 ml (or other applicable quantity that results in the user input ratio) of red ink, a signal or other indication may be provided to the ink container 210 to dispense 2 ml (or other applicable quantity that results in the user input ration) of green ink, and a signal or other indication may be provided to the ink container 220 to dispense 4 ml (or other applicable quantity that results in the user input ration) of blue ink. The ink dispensed by each of the ink containers 200, 210, and 220 may be dispensed and may traverse via paths 202, 212, and 222 respectively, which may be considered flow paths. Each of the paths 202, 212, and 222 may end at mixing container 230 such that the ink dispensed from the ink containers 200, 210, and 220 is dispensed into mixing container 230 via paths 202, 212, and 222, respectively. The red, green, and blue ink with a respective ratio of 3:2:4 may mix inside the mixing container 230 to produce a custom ink color. A mixing mechanism 231 may be included inside the mixing container 230 and may enable periodic mixing of the custom ink inside of the mixing container 230. For example, the mixing mechanism 231 may move when the mixing container 230 is moved or shaken, and may prevent the ink inside the mixing container 230 from settling.

According to an implementation, a user may input a desired custom color via an input device, such as the input device 240 of FIG. 2. According to this implementation, a user may either provide a desired color or select a desired color based on a spectrum of colors displayed to the user via the input device 240. Corresponding colored ink amounts may be determined based on the user's input such that ink from two or more of the ink containers 200, 210, and 220 may be dispensed based on the determination. For example, a user may input a desired color using input device 240. The input device 240, or other computing device, may determine a ratio of two or more colored inks that mix to result in the desired color. Based on the determination, the user-activated dispensers for the ink containers corresponding to those two or more colored inks may dispense the applicable ratio of colored ink to a mixing container.

According to implementations of the disclosed subject matter, a mixing station, such as the mixing station 1000 of FIG. 1 and 2000 of FIG. 2 may be placed on or may comprise a flat work surface with the ink containers placed on top of the flat work surface. A mixing station may be curved such that one or more ink containers surround a user as the user is using the mixing station. The mixing station may be covered by or may include a graphical display such as a drape, a hanging, a banner, or the like. Alternatively or in addition, a mixing station may include illumination such as, for example, LED lighting. As a specific example, the mixing station may include LED lighting that matches the color of ink that is contained within ink containers. Alternatively or in addition, a mixing station may include sound output. Sound may be output at any time and may be random or may be pre-configured. For example, sound may output when ink is dispensed from an ink container. Different sounds and/or illumination may be produced based on ink being dispensed from different ink containers.

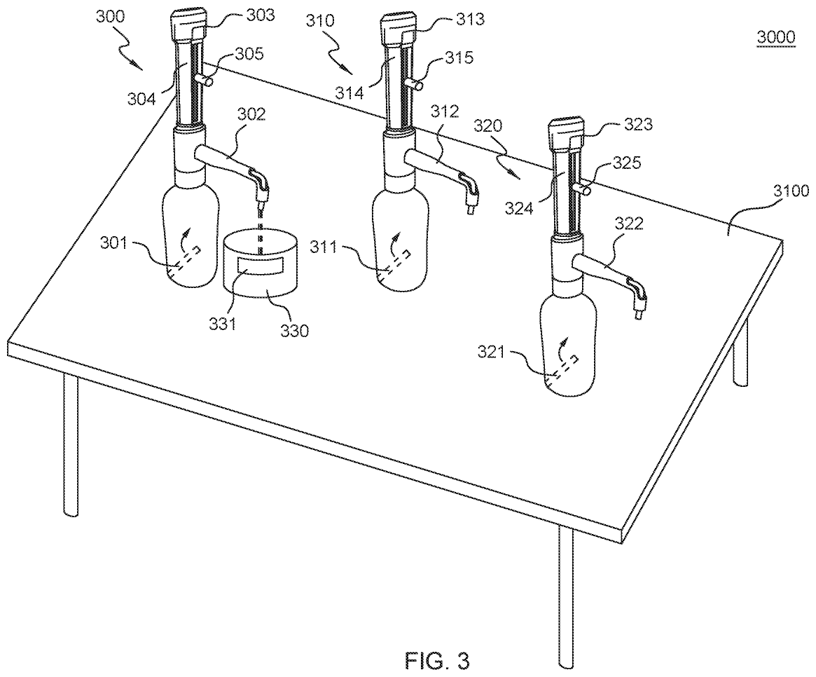

FIG. 3 shows an example mixing station 3000 comprising three ink containers 300, 310, and 320. The ink containers 300, 310, and 320 include a pump 303, 313, and 323 respectively. The pumps 303, 313, and 323 may be pressed automatically or may be manually pressed to facilitated the ink from the base of the respective containers through the spouts 302, 312, and 322 respectively. The shafts 304, 314, and 324 of the pumps 303, 313, and 323 may move down to facilitate the pump action. The shafts 304, 314, and 324 may move back up to their original positions based on pressure from a spring or other pressure exerted or biased on the shafts 304, 314, and 324. Amount selectors 305, 315, and 325 may be used to determine the amount of ink to be dispensed via each pump, based on the amount designated by the selector. These are preferably adjustable by a user. For example, amount selector 305 may be adjusted to select the amount, e.g., number of millimeters, of the ink contained in container 300 that should be dispensed from each pump (actuation) of the pump 303. For example, the amount selector may be adjusted to 3 ml for a first pump and to 2 ml for a different pump. The amount selectors may be pins configured to be placed in openings in the shafts, whereby setting a pin in a particular opening allows for movement corresponding to a particular amount of ink to be dispensed, It is also appreciated that dials or knobs may be used as the amount selectors, as well as any other mechanical devices that can set how much ink is dispensed per each actuation of a pump.

The ink containers 300, 310, and 320 may be mounted or placed on a work surface 3100. The ink containers 300, 310, and 320 may each contain a different ink color. For example, ink container 300 may include red ink, ink container 310 may include green ink, and ink container 320 may include blue ink. Each of the ink containers 300, 310, and 320 may include a mixing mechanism 301, 311, and 321 respectively. As shown, the mixing mechanisms 301, 311, and 321 may be located inside the ink containers 300, 310, and 320 and may rotate in the direction shown in FIG. 1. The rotation of the mixing mechanisms 301, 311, and 321 may mitigate the settlement of the ink contained in each respective ink container. A user may interact with pumps 303, 313, and/or 323 in order to dispense respective inks from the ink containers 300, 310, and 320, based on the amounts of inks selected via respective amount selectors 305, 315, and 325. For example, a user may press button 103 to dispense 1 ml of red ink from the ink container 300 into the mixing container 330, based on the amount selector 305 designating 1 ml. The user may subsequently press button 323 to displease 3 ml of blue ink from the ink container 320 into the mixing container 330, based on the amount selector 315 designating 3 ml. The 1 ml of red ink may mix with the 3 ml of blue ink to produce a custom ink color within the mixing station. The ink may be dispensed from the ink containers 100, 110, and 120 via dispensers 302, 312, and 322, respectively. As further disclosed herein, a label 331 may be generated and may include a custom color or other indicia (e.g., user name, unique title or name for color, etc.) for the custom ink and/or the custom formula for the custom ink within the mixing container 330. The label (e.g., printout) may be printed at the mixing station 3000 or may be provided to the user via an alternative mode such as via email or via transmission to a printer separate from mixing station 3000. The custom ink may be stored in a user's profile, as further discussed herein.

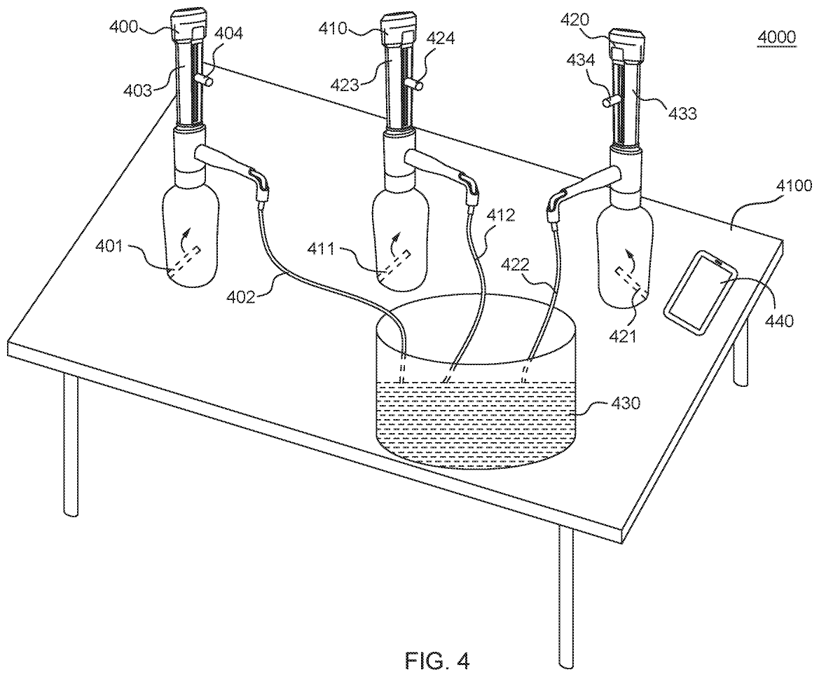

FIG. 4 shows another example mixing station 4000 comprising three ink containers 400, 410, and 420. The ink containers 400, 410, and 420 may be on a station 4100. The ink containers 400, 410, and 420 may each contain a different ink color. For example, ink container 400 may include red ink, ink container 410 may include green ink, and ink container 420 may include blue ink. Each of the ink containers 400, 410, and 420 may include a mixing mechanism 401, 411, and 421 respectively. As shown, the mixing mechanisms 401, 411, and 421 may be located inside the ink containers 400, 410, and 420 and may be attached to the walls of the ink containers. In this example, the walls of the ink containers 400, 410, and 420 may rotate such that the mixing mechanisms 401, 411, and 421 rotate along with the rotation of the walls. The rotation of the mixing mechanism 401, 411, and 421 may mitigate the settlement of the ink contained in each respective ink container. A user may interact with input device 240, which may be a mobile device, a tablet, or the like and may enable interaction via a touch screen.

Interaction with an input device 440 may cause ink to dispense from one or more of the ink containers 400, 410, and 420. For example, a user may input a ratio of 3:2:4 corresponding to 3 parts red, 2 parts green, and 4 parts blue via the input device 440. Based on the user input, a signal or other indication may be provided to ink container 400 to dispense 3 ml (or other applicable quantity that results in the user input ratio) of red ink, a signal or other indication may be provided to the ink container 410 to dispense 2 ml (or other applicable quantity that results in the user input ration) of green ink, and a signal or other indication may be provided to the ink container 420 to dispense 4 ml (or other applicable quantity that results in the user input ration) of blue ink. The ink dispensed by each of the ink containers 400, 410, and 420 may be dispensed and may traverse via paths 402, 412, and 422 respectively. Each of the paths 402, 412, and 422 may end at mixing container 430 such that the ink dispensed from the ink containers 400, 410, and 420 is dispensed into mixing container 430 via paths 402, 412, and 422, respectively. The red, green, and blue ink with a respective ratio of 3:2:4 may mix inside the mixing container 430 to produce a custom ink color. A mixing mechanism 431 may be included inside the mixing container 430 and may enable periodic mixing of the custom ink inside of the mixing container 430. For example, the mixing mechanism 431 may move when the mixing container 430 is moved or shaken, and may prevent the ink inside the mixing container 430 from settling.

Any input device described herein can be, for example, a computer, notebook, tablet, iPad, or other electronic device. The input devices or a hard-copy form may also be used and provided at a mixing station, allowing a user to track the quantities, types, ratios, and other information related to the ink mix used or selected by that customers. In this manner, the customer can track the various inks used and has a record of the amounts and types of ink used. The input devices are preferably provided with software (such as an application) that will create a user profile for a specific user, and store information about the user (such as demographic information) and the user's ink preferences, mixing history and/or saved ink mixes. In this manner, a user can track, for example, favorite ink mixes for later use, or to be experimented with at another time. A user can also enter custom names for their ink mixes, to be saved by the system.

According to implementations of the disclosed subject matter, ink containers such as the ink containers 100, 110, and 120 of FIGS. 1, 200, 210, and 220 of FIGS. 2, 300, 310, and 320 of FIG. 3 as well as 400, 410, and 420 of FIG. 4 may be made from any applicable material including, but not limited to, metal, plastic, or a combination thereof. Different ink containers may be colored differently such that, for example, the color of an ink container reflects the color of the ink inside the container. According to an implementation, an ink container may include a window such that a user may be able to see the color of the ink inside the ink container.

The ink inside different ink containers may be different colors. As an example, different ink containers may contain different base colored ink. The ink contained in the ink containers may be suitable for tattoos and may include, for example, carbon, antimony, arsenic, beryllium, calcium, lithium, selenium, and sulphur. The ink may include iron oxides (rust), metal salts, or plastics. Heavy metals used for colors may include, for example, mercury (red); lead (yellow, green, white); cadmium (red, orange, yellow); nickel (black); zinc (yellow, white); chromium (green); cobalt (blue); aluminium (green, violet); titanium (white); copper (blue, green); iron (brown, red, black); and barium (white). Metal oxides used include ferrocyanide and ferricyanide (yellow, red, green, blue). Organic chemicals used include azo-chemicals (orange, brown, yellow, green, violet) and naptha-derived chemicals (red). Carbon (soot or ash) is also used for black. Other elements used as pigments include antimony, arsenic, beryllium, calcium, lithium, selenium, and sulphur. The ink may contain one or more lighting agents such as, for example, lead or titanium.

According to implementations of the disclosed subject matter, a user-activated dispenser may be any applicable dispenser that dispenses ink from an ink container to a mixing container. As shown in the example mixing station 1000 of FIG. 1, the user-activated dispenser may be a spout, which dispenses ink from ink containers 100, 110, and 120 such that the ink traverses a distance prior to being deposited in, for example, a mixing container 130. As shown in an alternate example of mixing station 2000 of FIG. 2, the user-activated dispenser may be a tube, which dispenses ink from ink containers 200, 210, and 120 directly into mixing container 230 such that each ink container 200, 210, and 220, via a flow path.

Increments of a pre-selected amount of ink may be dispensed when an input device is receives a color input form a user. The pre-selected amount of ink may correspond to a specific quantity such as, for example, a number of millimeters. As an example, a user may press a button 103 of FIG. 1, such that 1 ml of ink is dispensed from the ink container 100. Each time the user presses the button 103, 1 ml of ink in ink container 100 may be dispensed. Alternatively, the pre-selected amount of ink may correspond to a ratio of first colored ink to other colored inks such that the ratio corresponds to the input provide by a user. For example, a user may use the input device 240 of FIG. 2 to input a ratio of colored ink such that the user inputs how much of a first, second, and third ink are to be dispensed. Accordingly, the amount of ink dispensed by containers based on the user's input, 200, 210, and 220 may reflect the ratio input by the user.

According to implementations disclosed herein, a mixing mechanism may be used to prevent ink inside an ink container and/or a mixing container from settling. A mixing mechanism may be any applicable mechanism that mitigates the settling of ink. A mixing mechanism may be one or more rods, bars, ball bearing, spheres, pebbles, mesh, mechanical component, electronic component, chemical, compound, or the like that mitigates the settlement of an ink.

A mixing mechanism may be in constant motion inside an ink container. For example, a mixing mechanism 101 such as a rod of FIG. 1 may constantly rotate inside ink container 100. The constant motion of the mixing mechanism 101 of the rod may cause ink to experience periodic motion and, thus, may mitigate the settling of ink inside the ink container 100.

A mixing mechanism may be powered using a battery or via an electrical connection. For example, an ink container may include a battery that powers the movement of a mixing mechanism. Alternatively or in addition, an electrical connection may provide current to a mixing mechanism such that the mixing mechanism operates using the current. One or more ink containers as well as the mixing container may include a mixing mechanism. Mixing mechanisms for different ink containers and/or the mixing container may be the same or may be different than each other, or a combination thereof. For example, the ink containers at a mixing station may include battery powered mixing containers that rotate constantly inside of an ink container. The mixing container may have one or more pellets inside it which move when the mixing container is physically moved.

According to an implementation of the disclosed subject matter, a sterile environment may be maintained within ink containers, activated dispensers, and/or mixing containers. A sterile environment may be necessary to protect the ink from contamination from microorganisms.

According to an implementation of the disclosed subject matter, an ink container may contain a thinning solution and/or a thickening solution. The thinning solution and/or thickening solution may modify the consistency of a custom ink, such that if thinning solution and/or thickening solution are mixed with one or more inks, in a mixing container, the resulting custom ink's consistency is determined, at least in party, by the thinning and/or thickening solution. A desired consistency may be obtained as a result of thinning and/or thickening solution. As an example, the ink container 100 of FIG. 1 may contain first colored ink, the ink container 110 may contain second colored ink, and the ink container 120 may contain a thinning solution. A user may provide an input such that a portion of the first colored ink is deposited into mixing container 130, a portion of the second colored ink is deposited into mixing container 130 and the thinning solution is deposited into the mixing container 130. The resulting custom ink in the mixing container 130 may be thinner than if the thinning solution from ink container 120 was not deposited. According to an implementation, a user may provide a desired viscosity via an input device such as the input device 240 of FIG. 2. Based on the desired viscosity, the system may determine the amount of thinning solution and/or thickening solution and may displease such amount into a mixing container. According to an implementation, a thinning solution and/or thickening solution may be mixed with one or more inks prior to the ink entering a mixing container.

According to an implementation, an amount of thinning solution and/or thickening solution may be determined based on the user selected colored ink. Notably, the system may determine an optimal amount of thinning solution and/or thickening solution based on properties of the selected inks to maintain an optimal viscosity based on the ink selection.

According to an implementation, a mixing container may be sealed after colored ink is deposited into the mixing container. The mixing container may be configured such that a sealing mechanism is provided along with the mixing container for sealing the mixing container. For example, the mixing container may include flap, which is covered by non-stick tape. Removal of the non-stick tape may convert the flap to become adhesive such that the flap seals the opening in a mixing container to seal close the mixing container.

According to an implementation, a label may be generated based on the custom colored ink. The label may be configured to affix to a mixing container or may be a standalone label such as a pamphlet or other tangible item. The label may include the custom formula for the custom colored ink inside the mixing container. Alternatively or in addition, the label may include a name for the ink inside the mixing container. Alternatively or in addition, the label may include user provided content such as, for example, the user's name, a logo, an image, text, or the like. An input device or other component may provide the custom formula or other content to be included in the label. For example, a user may input a custom color via the input device 240 of FIG. 2. The input device 240 may provide the custom formula for the custom color (e.g., amount or ratio of each color ink that is included in the creating the custom color) to a label generating printer. A label that includes the custom formula may be printed and applied onto a mixing container that includes the custom ink. Alternatively or in addition, a label or other content may be electronically transmitted to a user or other entity, For example, a user may receive an email notification, which includes the custom formula for the custom color ink that the user created using a mixing station.

According to an implementation, a user may select amounts of different colored inks via an input (e.g., button press) and a representative sample of the different colored inks mixed together may be provided to the user prior to the actual amount of ink is dispensed. The representative sample may be a digital sample or a physical sample, For example, the user may select three different colored inks to be dispensed. The user may be provided a representation of the three colors mixed based on the proportions provided by the user via, for example, a monitor. According to this implementation, the user may be able to visualize the mixed colors via the display, prior to committing to the mixed color and prior to the system dispensing the three colors. Further, a display or a three-dimensional representation (e.g., a hologram) may be provided to display the mixed color. For example, a the representation of the mixed color may be provided overlaid on a body part so that a user can visualize the mixed color as a tattoo prior to committing to the mixed color. The mixed color may be provided to the user in the shape of a tattoo. According to an implementation, the shape of a tattoo may be determined based on the user's profile (e.g., tattoos or body parts previously provided by the user).

According to an implementation, a user profile may be created and information related to the user profile may be stored locally or at a remote location (e.g., a server or database). The user profile may be include user information such as the user's username, password, address, email, etc., and/or may include current or previous custom color formulations. For example, a user may create a custom color and the amount/ratios of two or more inks and/or thickening or thinning agent amounts related to that custom color may be stored in the users profile such that the system or the user may be provided access to them at a current or future time. For example, the user may be provided a custom color stored at a previous time. The user may then chose to modify that custom color by adjusting a first color to be more prominent. A new custom color may be stored in the user's profile based on the adjustment.

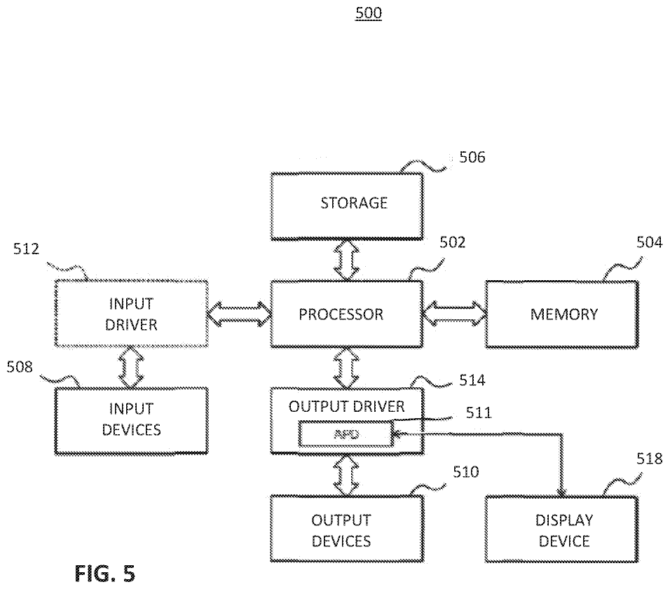

FIG. 5 is a block diagram of an example system 500 in which one or more features of the disclosure can be implemented. The system 500 could include one of, but is not limited to, for example, a computer, a handheld device, a set-top box, a mobile phone, a tablet computer, a wearable watch, a holographic device, a virtual reality device, an augmented reality device, or other computing device. The system 500 may be used to receive color based input from users, determine mixing proportions (e.g., based on a target color), determine an amount of thinning solution or thickening solution, as disclosed here. The system 500 includes a processor 502, a memory 504, a storage 506, one or more input devices 508, and one or more output devices 510. The system 500 also includes one or more input drivers 512 and one or more output drivers 514. Any of the input drivers 512 are embodied as hardware, a combination of hardware and software, or software, and serve the purpose of controlling input devices 512 (e.g., controlling operation, receiving inputs from, and providing data to input drivers 512). Similarly, any of the output drivers 514 are embodied as hardware, a combination of hardware and software, or software, and serve the purpose of controlling output devices 514 (e.g., controlling operation, receiving inputs from, and providing data to output drivers 514). It is understood that the system 500 can include additional components not shown in FIG. 5.

In various alternatives, the processor 502 includes a central processing unit (CPU), a graphics processing unit (GPU), a CPU and GPU located on the same die, or one or more processor cores, wherein each processor core can be a CPU or a GPU. In various alternatives, the memory 504 is located on the same die as the processor 502, or is located separately from the processor 502. The memory 504 includes a volatile or non-volatile memory, for example, random access memory (RAM), dynamic RAM, or a cache.

The storage 506 includes a fixed or removable storage, for example, without limitation, a hard disk drive, a solid state drive, an optical disk, or a flash drive. The input devices 508 include, without limitation, a keyboard, a keypad, a touch screen, a touch pad, a detector, a microphone, an accelerometer, a gyroscope, a biometric scanner, an eye gaze sensor 530, or a network connection (e.g., a wireless local area network card for transmission and/or reception of wireless IEEE 802 signals). The output devices 510 include, without limitation, color sample, a display, a speaker, a printer, a haptic feedback device, one or more lights, an antenna, or a network connection (e.g., a wireless local area network card for transmission and/or reception of wireless IEEE 802 signals).

The input driver 512 and output driver 514 include one or more hardware, software, and/or firmware components that are configured to interface with and drive input devices 508 and output devices 150, respectively. The input driver 512 communicates with the processor 502 and the input devices 508, and permits the processor 502 to receive input from the input devices 508. The output driver 514 communicates with the processor 502 and the output devices 150, and permits the processor 502 to send output to the output devices 150. The output driver 514 includes an accelerated processing device (“APD”) 511 which is coupled to a display device 518. In some implementations, display device 518 includes a desktop monitor or television screen. In some implementations display device 518 includes a head-mounted display device (“HMD”), which includes screens for providing stereoscopic vision to a user. In some implementations the HMD also includes an eye gaze sensor for determining the direction in which the eye of a user is looking.

FIG. 6 shows an example input device 600. Input device 600 may be the same as or similar to input device 240 of FIG. 2, as disclosed herein. As shown in FIG. 6, input device 600 may include fields for user input including a name field 602 and a custom ink color name field 604. Multiple ink color options such as colors 610, 620, and 630 may be visually represented via input device 600. A user may interact with buttons 612, 622, and 632 to provide color selection input related to colors 610, 620, and 630, respectively. For example, a user may use color selection button 612 to increase or decrease the amount of color 610. use color selection button 622 to increase or decrease the amount of color 620, and/or use color selection button 622 to increase or decrease the amount of color 630. The user may use any applicable input component such as a touch screen, keyboard, Bluetooth connected device, mouse, touchpad, gesture, or the like, to provide field inputs and/or color selections.

The amount of a given color may be provided to the user via the input device 600. As shown in FIG. 6, color amount indicator 614 may correspond to the amount of ink of color 610 is currently selected by the user. Similarly, color amount indicator 624 may correspond to the amount of ink of color 620 is currently selected by the user and color amount indicator 634 may correspond to the amount of ink of color 630 is currently selected by the user. It will be understood that color selection buttons (e.g., 612, 622, and 632) may be used to provide an amount of ink for a color, a ratio, or any other value or distinguishing attribute that can be used to output ink.

A user may finalize the inputs including the name field 602, custom ink color name field 604, and color selections by selecting the submit button 650. The input device 600 may be an input device 508 of FIG. 5 such that the name field 602, custom ink color name field 604, and color selections are provided to the input driver 512 and then processed by processor 502. The processor 502 may provide the name field 602, custom ink color name field 604, and color selections to the storage component 506 such that they are stored (e.g., associated with a user profile). The processor may also provide color selections to output devices 510 which may include ink containers (e.g., ink containers 100, 110, and 120 of FIG. 1, ink containers 200, 210, and 220 of FIG. 2, or ink containers 300, 310, and 320 of FIG. 3), via one or more output drivers 514.

The output devices 510 (e.g., ink containers) may output colored ink in accordance with the techniques, systems, and devices disclosed herein, based on the color selections input by a user via interactive device 600. The colored ink output by the output devices 510 (e.g., ink containers) may be mixed within a mixing container (e.g., mixing container 130, 230, or 330 of FIGS. 1, 2, and 3 respectively).

The figures provided herein are provided as an example only. At least some of the elements discussed with respect to these figures can be arranged in different order, combined, and/or altogether omitted. It will be understood that the provision of the examples described herein, as well as clauses phrased as “such as,” “e.g.”, “including”, “in some aspects,” “in some implementations,” and the like should not be interpreted as limiting the disclosed subject matter to the specific examples.

Having described the invention in detail, those skilled in the art will appreciate that, given the present disclosure, modifications may be made to the invention without departing from the spirit of the inventive concepts described herein. Therefore, it is not intended that the scope of the invention be limited to the specific embodiments illustrated and described.

What is claimed is:

1. A mixing station for a customizable tattoo ink comprising:
a work surface;
a plurality of ink containers disposed on the work surface, wherein each ink container comprises a user-activated dispenser, each user-activated dispenser configured to dispense ink in a selected amount related to an activation of the dispenser;
an input device configured to receive user input from a user related to a creation of a custom ink;
a first ink container from the plurality of ink containers comprising a first colored ink and a first mixing mechanism configured to agitate the first colored ink formed from one of more inks;
a second ink container from the plurality of ink containers comprising a second colored ink and a second mixing mechanism configured to agitate the second colored ink; and
a mixing container configured to receive at least a selected quantity of the first colored ink from the first container based on the user input, and a selected quantity of the second colored ink from the second container based on the user input, and to store the custom ink.

2. The mixing station of claim 1, wherein the dispenser comprises a pump and a spout.

3. The mixing station of claim 1, wherein the dispenser comprises a tube providing a flow path from an ink container to the mixing container.

4. The mixing station of claim 1, wherein the mixing container comprises a third mixing mechanism configured to agitate the custom ink.

5. The mixing station of claim 1, wherein the ink container and dispenser maintain a sterile environment.

6. The mixing station of claim 1, wherein the plurality of ink containers are configured to dispense ink in increments of at least one milliliter.

7. The mixing station of claim 1 further comprising:
a third ink container comprising a thinning solution; and
a fourth ink container comprising a thickening solution.

8. The mixing station of claim 7, wherein the input device is configured to receive a selected desired consistency from a user.

9. The mixing station of claim 7, wherein the input device is configured to determine a custom formula, wherein the custom formula comprises at least two of the amount of the first color ink, the amount of the second color ink, the amount of the thinning solution, or the amount of the thickening solution.

10. The mixing station of claim 9, wherein the mixing station is configured to provide the custom formula via at least one of a printout or an electronic transmission.

11. The mixing station of claim 10, wherein the mixing station is configured to print a label comprising the custom formula.

12. The mixing station of claim 1, wherein the input device comprises at least one of a computer, a tablet, a mobile device, a wireless device, a touch screen, a keyboard, a mouse, a lever, a button, a dial, a knob, or a switch.

13. A method comprising:
activating a first mixing mechanism inside a first ink container;
activating a second mixing mechanism inside a second ink container;
providing an input via an input device wherein the input comprises at least one of a desired color, a first color amount, a second color amount, or a ratio between a first color and a second color.
dispensing, from the first ink container, an amount of first ink based on the input via the input device;
dispensing, from the second ink container, an amount of second ink based on the input via the input device;
receiving the amount of first ink and the amount of second ink at a mixing container;
mixing the amount of first ink and the amount of second ink at the mixing container to create a custom ink;
sealing the mixing container; and
providing a label corresponding to custom ink.

14. The method of claim 13, further comprising:
dispensing a thinning solution from a third container; and
dispensing a thickening solution from a fourth container.

15. The method of claim 14, wherein the thinning solution and the thickening solution modify a consistency of custom ink in the mixing container.

16. The method of claim 14, further comprising determining a custom formula comprising at least two of the amount of the first ink, the amount of the second ink, the amount of the thinning solution, or the amount of the thickening solution.

17. The method of claim 13, wherein the first mixing mechanism and the second mixing mechanism are independent of each other.
